(19) United States
(12) Patent Application Publication
(10) Pub. No.: US 2006/0194721 A1
(43) Pub. Date: Aug. 31, 2006

(54) Tissue regeneration

(76) Inventor: Shelly Jane Allen, (US)

Correspondence Address: VINSON & ELKINS, L.L.P., 1001 FANNIN STREET, 2300 FIRST CITY TOWER, HOUSTON, TX, 77002-6760, US

(21) Appl. No.: 10/545,381

(22) PCT Filed: Feb. 12, 2004
(86) PCT No.: PCT/GB04/00534

§ 371 Date: Nov. 2, 2005

(30) Foreign Application Priority Data
Feb. 13, 2003 (GB) 03033362.8

(51) Int. Cl. A61K 38/18; A61K 38/39

(52) U.S. Cl. 514/12

(57) ABSTRACT

A biocompatible, biodegradable composition for encouraging controlled growth, regeneration or repair of biological tissue or cells, the composition comprising a scaffold, formed from biodegradable and biocompatible material, and a receptor for a growth factor, or a growth factor-binding fragment or homologue thereof, located at or adjacent a surface of the scaffold. The tissue or cells are preferably neuronal, in which case the receptor is preferably a tyrosine receptor kinase (Trk), or a neurotrophin-binding fragment or homologue thereof. Such a composition may include one or more types of neurotrophin bound to the Trk or fragment or homologue thereof.

This invention relates to the regeneration and repair of biological tissues. In particular, although not exclusively, the invention concerns the use of biocompatible compositions for encouraging growth, regeneration and/or repair of neuronal tissue.

Nerve repair using autograft material has several shortcomings, including donor site morbidity, inadequate return of function, and aberrant regeneration. Alternatives to autografts have been sought for use in bridging neural gaps. Many entubulation materials have been studied, although with generally disappointing results in comparison with autografts. Recently, peripheral nerve research has focused on the generation of synthetic nerve guidance conduits that might overcome these problems. In various laboratories, synthetic biodegradable polymers, which are removed from the engineered tissues by hydrolysis and dissolution of breakdown products, have been used in conjunction with Schwann cells to create a superior prosthesis for the repair of branched peripheral nerves (Hadlock et al (1998) Arch Otolaryngol Head Neck Surg, 124: 1081; Hadlock et al (2000) Tissue Eng, 6: 119; Bryan et al (2000) Tissue Eng, 6: 129). The functioning of tissues such as nerves and blood vessels is dependent on the controlled orientation of cells; for many tissue cell types, the spatial organisation is required to ensure that cell-to-cell interactions occur. Synthetic materials may be engineered such that they mimic cellular microenvironments encountered during natural development. For example, biodegradable polymer surfaces can be engineered to present peptides containing the amino acid sequence arginine-glycine-aspartate (RGD). This sequence binds to integrin receptors on cell surfaces, inducing cell adhesion, spreading and intracellular signalling, and hence mimicking cell-to-extracellular matrix interactions.

There are a range of techniques by which biomolecules can be immobilized on surfaces with micron-scale precision. These techniques include lithographic methods, which use patterned masks to restrict the location of interactions between a beam of light, ions or electrons and a surface, and micro-contact printing techniques. These techniques, however, can restrict the types of ligands and surfaces that can be patterned.

As shown in WO99/36107, it is possible to generate micron-scale patterns of biotinylated ligands on the surface of a biodegradable block copolymer, achieving control of biomolecule deposition with nanometer precision. This is confirmed by molecular resolution of protein molecules on the patterned surfaces using atomic force microscopy. This system has been tested in cultured bovine aortic endothelial cells and PC12 nerve cells and shows spatial control over cell development. Neurite extension of PC12 cells, on the polymer surface, can be directed by pattern features composed of peptides containing the IKVAV sequence (Patel et al (1998) FASEB J, 12: 1447; Cannizaro et al (1998) Biotechnol Bioeng, 58: 529).

The polymer used in the above system is generally a block copolymer of biotinylated poly(ethylene glycol) (PEG) with poly(lactic acid) (PLA) which uses the high affinity coupling of biotin-avidin as post fabrication surface engineering. These poly(esters) are susceptible to acid catalysed hydrolysis and are thus biodegradable. Biodegradability rate may be controlled and thus the polymers may be used for the controlled delivery of therapeutic agents. The pH-sensitivity of a related class of polymers, the poly (orthoesters), has also been studied for this purpose (Leadley et al (1998) Biomaterials, 19: 1353-60).

However, there is a drawback to all of the methods used previously. Neurones require, for their maintenance and neurite outgrowth, the presence of various growth factors. Experiments carried out under cell culture conditions are generally in the presence of foetal calf serum or added growth factors. However, under normal conditions, in the body, levels of circulating growth factors are too low to be effective for nerve regeneration.

Nerve growth factor (NGF) is one of a family of neurotrophins; other family members include brain-derived neurotrophic factor (BDNF), neurotrophin-3 (NT3) and neurotrophin-4 (NT4; sometimes referred to as NT4/5 or NT5). All of the neurotrophins bind to a common receptor, p75NGFR. Specificity is defined through their interaction with tyrosine receptor kinases (Trk) the Kd of which interaction is approximately 10⁻¹⁰-10⁻¹¹M.

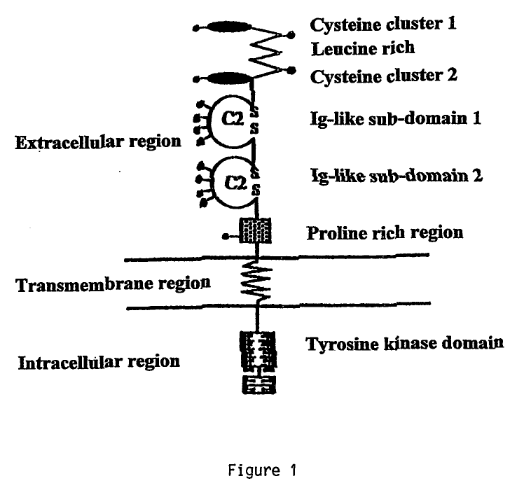

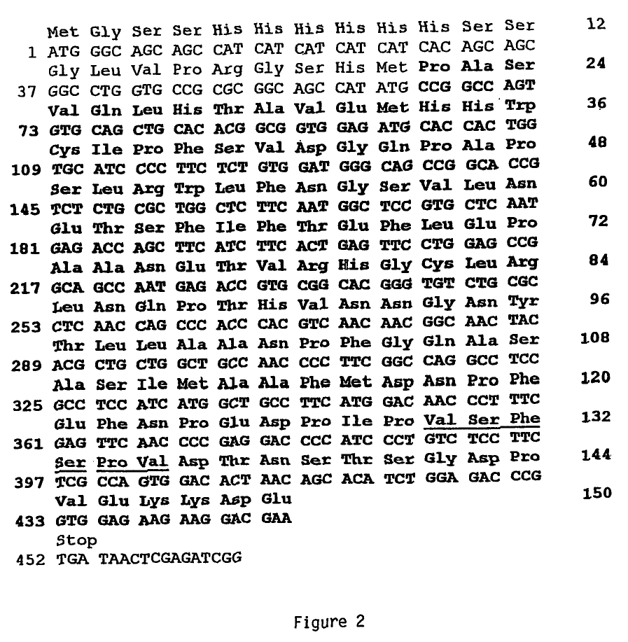

The properties of TrkA are described in WO99/53055. A schematic representation of the TrkA structure is appended as FIG. 1. The nucleotide sequence and derived amino acid sequence of the immunoglobulin (Ig)-like binding domain 2 (TrkAIg2) are appended as FIG. 2.

NGF binds to TrkA, BDNF and NT4 bind to TrkB and NT-3 binds to TrkC and an alternatively spliced version of TrkA which has a six amino acid insert VSFSPV (underlined in FIG. 2) in its Ig-like binding domain 2.

The majority of peripheral and spinal nerves require the presence of one or more of the neurotrophins for survival. Recent studies indicate that neurotrophic factors play a significant role in helping the developing and adult nervous system survive after axotomy. Before regenerating, neurones need to first survive axotomy. Neurotrophins rescue immature (Diener and Bregman (1994) Neuroreport, 5: 1913) and mature (Shibayama et al (1998), J Comp Neurol, 390: 102) axotomised central nervous system (CNS) neurones from retrograde cell death. Axotomy of neurones in the peripheral nervous system (PNS) frequently leads to upregulation of regeneration-associated genes, which assist in regeneration. Only transient increases in these genes occur in the CNS after axotomy, close to the cell body, but not when the lesion is more distal. Prolonged induction of regeneration-associated genes may be required for regeneration in this situation. Neurotrophins increase the expression of regeneration associated genes (e.g. c-Jun, GAP-43, Ta1tubulin). In cultured adult dorsal root ganglion cells (DRG), types of axon growth (arborization or elongation) depend on different patterns of gene expression (Smith and Skene (1997) J Neurosci, 17: 646). BDNF, for instance, enhances GAP-43, supporting the branching process.

Some researchers have grafted cells that are genetically modified to secrete growth factors such as NGF at the injury site (Grill et al (1997) Exp Neurol, 148: 444), whilst others have looked at the release profile of NGF, co-encapsulated with ovalbumin, from biodegradable polymeric microspheres such as those prepared from PLGA 50/50, PLGA 85/15, PCL and a blend of PCL/PLGA 50/50 (Cao and Schoichet (1999) Biomaterials, 20: 329). NGF was found to be released and bioactive for at least 3 months.

Other researchers have tried to transplant foetal cells into the site of injury in the spinal cord. The remodelling of axonal projections in vivo after spinal cord injury and transplantation is regulated by the availability of neurotrophic factors. In the adult, exogenous NGF increases the growth of axotomised dorsal root axons into the spinal cord (Oudega and Hagg (1996) Exp Neurol, 140: 218; Oudega et al (1994) Exp Neurol, 129: 194). After spinal cord hemisection and foetal cord transplantation in the adult, the exogenous administration of BDNF, NT3 and NT4 increased the amount of supraspinal growth into the foetal transplant. Ciliary derived neurotrophic factor (CNTF) failed to do this. BDNF and NT3 also support the regrowth of brainstem fibres into Schwann cell grafts placed into thoracic level lesions in the adult rat (Xu et al (1995) Exp Neurol, 134: 261). Cells modified to secrete NGF and NT3, transplanted into spinal cord, influence the axonal growth of spinally projecting neurones (Tuszynski et al (1996) Exp Neurol, 137: 157; Grill et al (1997) J Neurosci, 17: 5560), and are associated with an improvement in motor function (Grill et al (1997) J Neurosci, 17: 5560).

NT3 and BDNF also induce oligodendrocytic proliferation and myelination of regenerating axons in the spinal cord after contusion injury (McTigue et al (1998) J Neurosci, 18: 5354).

Recent studies have shown that, in addition to acute injury, neurotrophins may assist regrowth in chronic injury (Ye and Houle (1997) Exp Neurol, 143: 70; Houle et al (1997) Restorative Neurol Neurosci, 10: 205; Houle and Ye (1997) Neuroreport, 8: 751).

The prior art described above, whilst indicating the importance of neurotrophic growth factors in neuronal survival, repair and regeneration, and the possibility of growing neuronal tissue on biodegradable polymer scaffolds in vitro, leaves open the question of how neuronal cells can be efficiently grown under conditions of low prevailing growth factor concentration. Furthermore, control of the rate and direction of neuronal growth is not addressed.

It is an object of the present invention to provide products and their uses which are capable of supporting the growth, regeneration and/or repair of neuronal and other tissues and cells and which do not suffer to such an extent from the problems identified in relation to the prior art.

Accordingly, a first aspect of the invention provides a biocompatible, biodegradable composition for encouraging controlled neuronal growth, regeneration or repair, the composition comprising a scaffold, formed from biodegradable and biocompatible material, and a tyrosine receptor kinase (Trk), or a neurotrophin-binding fragment or homologue thereof, located at or adjacent a surface of the scaffold.

The term ‘biodegradable’ as used herein means capable of being broken down, fragmented and/or dissolved on exposure to physiological or physiological-type media at pH6.0 to 8.0 and a temperature of 25 to 37° C. The period over which such breaking down, fragmentation and/or dissolution occurs will depend upon the intended application of the composition. Typical periods will be less than or about five years, more often between one week and one year. The term ‘biocompatible’ as used herein means that the material to which the term refers, and its biodegradation products, are not unacceptably toxic, immunogenic, allergenic or pro-inflammatory when used in vivo. The term ‘scaffold’ as used herein refers to any structure upon, within or through which cells may be supported for growth, regeneration or repair.

Preferably, the composition of the invention will include one or more types of neurotrophin bound to the Trk or Trk fragment or homologue. The neurotrophins may be selected from nerve growth factor, brain-derived neurotrophic factor, neurotrophin-3 and neurotrophin 4.

The Trk of the composition may be TrkA, B or C, an alternatively spliced version thereof, a pan-Trk (i.e. a Trk which is capable of binding all the neurotrophins), a functional homologue of a Trk or a combination of Trk types. Fragments of Trk homologues, and homologues of Trk fragments, are also included. In preferred embodiments, the neurotrophin-binding fragment of the Trk comprises an immunoglobulin (Ig)-like sub-domain, preferably the Ig-like sub-domain 2 of TrkA (TrkAIg2 or TrkAIg2.6, shown in FIG. 2 as amino acids 22 to 150, with the six amino acids 130 to 135 only present in the TrkAIg2.6 splice variant). Alternatively, or additionally, the neurotrophin-binding fragment of the Trk may comprise both Ig-like sub-domains of TrkA (TrkAIg1,2). Such fragments of Trk A preferably also include the proline-rich region. When the Ig-like sub-domain 2 of TrkA is employed, either alone or with the Ig-like sub-domain 1, it preferably includes the amino acid insert VSFSPV (TrkAIg2.6, the insert is shown as amino acids 130 to 135 in FIG. 2). The neurotrophin-binding fragment of the Trk may comprise, or may consist of, the entire sequence shown in FIG. 2 (TrkAIg2.6-6His).

When the composition includes one or more types of neurotrophin, it is preferred that, if TrkA or a neurotrophin-binding fragment thereof is used, the neurotrophin is selected from NGF and NT3. If TrkB or a neurotrophin-binding fragment thereof is used, the neurotrophin is preferably selected from BDNF and NT4. If TrkC or a neurotrophin-binding fragment thereof is employed, NT3 is preferred.

In some embodiments of the present invention, the composition also includes one or more extracellular matrix components located at or adjacent a surface of the scaffold. These extracellular matrix components may comprise peptides containing the sequences RGD, YIGSR and/or IKVAV in order to encourage integrin or other cell-surface receptor-mediated neurone extension and growth factor responses.

Cells cultured upon predominantly hydrophilic biomaterials such as PLA-PEG-biotin require the additional presence of extracellular matrix molecules to adhere the cells to the surface.

Such extracellular matrix molecules include collagens, proteoglycans, elastin, hyaluronic acid and glycoproteins such as fibronectin (FN), vitronectin (VN), and laminin (LN). Short peptide domains found along these molecules are responsible for interacting with cell-surface adhesion receptors known as integrins. Binding of these receptors facilitates not only cell adhesion, but also triggers intercellular events such as migration, spreading and phenotypic expression. Although the whole extracellular molecule can be used in combination with a growth factor modified surface, intact adhesion molecules typically interact with a wide range of cell types with varying degrees of specificity. It may therefore be preferable to employ the short isolated peptide sequences, in order to create materials that specifically interact with targeted cell types to produce pre-defined responses.

Example integrin-binding peptide sequences include the ubiquitous Arginine-glycine-aspartic acid (RGD) sequence, which interacts with most cell types, and the Isoleucine-lysine-valine-alanine-valine (IKVAV), Leucine-arginine-glutamic acid (LRE), and Tyrosine-Isoleucine-glycine-serine-arginine (YIGSR) fragments, which are isolated from laminin and have been demonstrated to facilitate neuronal development. These peptide sequences may be used in combination, and their activity enhanced by using flanking peptide sequences to improve sequence accessibility.

In designing adhesion-peptide-modified surfaces, the surface concentration must be optimised. A minimum density of adhesion ligand is necessary for cell adhesion and migration, and high densities of peptide will inhibit cellular migration due to the strength of the adhesion (Huttenlocher, Sandborg, Horwitz (1995) Adhesion in cell migration. Curr. Opin. Cell Biol., 7: 697-706). An intermediate level of attachment force is therefore required to induce maximal migration rates (Schense, Hubbell (2000) Three-dimensional migration of neurites is mediated by adhesion site density and affinity. J. Biol. Chem., 275: 6813-6818; Palecek, Loftus, Ginsberg, Lauffenburger, Horwitz (1997) Integrin-ligand binding properties govern cell migration speed through cell-substratum adhesiveness. Nature, 385: 537-540).

The material of the scaffold is preferably a biodegradable and biocompatible polymer. The biodegradable and biocompatible polymer may be selected from: polyhydroxy acids such as polyhydroxybutyric acid, poly (lactic acid), poly (glycolic acid), poly (ε-caproic acid), poly (ε-caprolactone), polyanhydrides, polyorthoesters, polyphosphazenes and polyphosphates; polysaccharides such as hyaluronic acid; proteins such as collagen; poly (amino acids); poly (pseudo amino acids); and copolymers prepared from the monomers of any of these polymers. Polymers of lactic acid or glycolic acid, or copolymers of these monomers, are preferred. Particularly preferred are block copolymers of any of the above polymers with a poly(alkylene glycol), such as poly(ethylene glycol) (PEG). Most preferred are block copolymers of PEG with poly(lactic acid), poly(glycolic acid) or poly(lactic-co-glycolic) acid. The properties and advantages of these various polymers may be found in WO99/36107.

The Trk or fragment thereof may be located at or adjacent the surface of the scaffold, and more preferably at the end of a poly(alkyleneglycol) chain when block copolymers comprise the scaffold, by any means compatible with the biocompatible, biodegradable material and the Trk. Such means may include covalent attachment, adsorption or physical entrapment. It is preferred, however, that the Trk or fragment is attached to or adjacent the surface by means of one or more specific molecular interactions. By ‘specific molecular interactions’ is meant interactions between two or more binding components with at least 100-fold higher affinity, preferably at least 500-fold, at least 1000-fold or at least 2000-fold higher affinity, than that of the interaction between one of those binding components and other molecules which it may encounter, e.g. in cell culture or in vivo. The one or more specific molecular interactions which attach the Trk to or adjacent the surface of the scaffold preferably take place between one or more anchor molecules bound to or adjacent the scaffold surface and one or more tag molecules bound to the Trk or fragment.

The anchor and tag molecules may be the same or different. In certain embodiments, the anchor is an antibody or fragment thereof and the tag is the corresponding antigen or hapten, or vice versa. Preferably, the tag is biotin and the anchor is avidin or streptavidin, or vice versa Most preferably, an adapter molecule is also used which is capable of simultaneously binding to both the tag and the anchor. In such a case, both the tag and the anchor may be the same. In preferred embodiments, both the tag and the anchor are biotin and the adapter is avidin or streptavidin (avidin and streptavidin have a valency of 4 in their binding to biotin).

It will be appreciated that only one specific molecular interaction need be employed in the attachment of a Trk or fragment to or adjacent the surface of the scaffold in order for the composition to benefit from the advantages associated with specific molecular interactions. Thus, any other molecular interactions (e.g. between the anchor and an adapter molecule when the tag binds to the adapter by means of a specific molecular interaction) need not be specific.

Methodologies suitable for the covalent attachment of the anchor molecule to or adjacent the scaffold surface and of the tag molecule to the Trk or fragment are well known in the art and reference may be made to WO99/36107 and references cited therein. The composition of the present invention preferably has a tubular scaffold, the regeneration of the neurones preferably taking place along the lumens of the tubular structure. The Trk or fragment may be located on the luminal wall by flowing a solution of the Trk or fragment through a scaffold previously treated so as to be capable of binding the Trk or fragment. Thus, in the case of specific molecular interactions, the scaffold may previously have been treated such that the luminal walls are labeled with anchor molecules, the Trk in solution being labeled with tag molecules. If an adapter molecule is employed, this is presented to the scaffold before the tagged Trk.

Any additional components to be located on or adjacent the scaffold surface may be attached in the same manner as the Trk or fragments. When the composition includes one or more neurotrophins, these are introduced to the scaffold either bound to the Trk or fragment, or as a separate step following prior location of the Trk or fragment. In each case where a Trk, tag, adapter, anchor, neurotrophin or any other component of the composition is introduced to the scaffold in solution, it is generally useful to introduce an excess to ensure adequate loading of binding sites. The excess, some of which may, of course, have become non-specifically bound to the scaffold or other components, may then be flushed out. The preparation of surfaces in a manner similar to those which may be used in the present invention is described in WO99/36107. In particular, the patterning of Trk on or adjacent the surface of the scaffold may be achieved using methods analogous to those used in WO99/36107.

The composition may also include growing, regenerating or repairing nerve cells, or nerve cell progenitors or pluripotent stem cells.

The compositions of the present invention have the advantage that they allow a ready supply of neurotrophins to be made available for neuronal uptake. The neurotrophins are non-covalently bound to the scaffold and hence are releasable for use by neurones. This provides neurotrophic support for the neurones in a way not envisaged previously. Furthermore, and particularly in those embodiments where a spatially arranged, or patterned, location of Trk or fragments is employed, a directional neuronal extension may be achieved. The composition of the invention may be used, either in vitro or in vivo both as a sequesterer of neurotrophins for subsequent supply to neurones (in which case the composition may be employed with few or no neurotrophins bound initially) and as a source of neurotrophins for neurones (in which case the composition may include a higher proportion of neurotrophin-bound Trk molecules or fragments).

The levels of circulating neurotrophins are too low to support survival. Neurotrophins are normally released from innervated tissues and are internalised by neurones after binding to Trk receptors. This complex is then transported to the cell body where it is thought to exert its cell survival effects. In order to regenerate nerves in vivo it will be necessary to provide a local supply of neurotrophins. The present invention allows the neurotrophins to be supplied non-covalently bound to the scaffold via the Trk molecule or fragment.

In a second, and related, aspect of the invention there is provided the composition of the first aspect for use in therapy.

In a third aspect there is provided the use of a Trk, or a neurotrophin-binding fragment or homologue thereof, in the preparation of a medicament for encouraging nerve growth, regeneration or repair, the medicament comprising a scaffold formed from a biodegradable and biocompatible material, and the Trk or fragment or homologue being located at or adjacent a surface of the scaffold.

The invention also provides, in a fourth aspect, a method of encouraging nerve growth, regeneration or repair, the method comprising contacting a composition according to the first aspect of the invention with a source of neurotrophins so as to form Trk-neurotrophin complexes on or adjacent the surface of the scaffold, contacting the composition with a stem cell, nerve progenitor cell, neuronal cell or tissue and allowing the stem cell, nerve progenitor cell, neuronal cell or tissue to grow, regenerate or repair upon or adjacent the surface of the scaffold.

000The method of the fourth aspect may be carried out in vivo or in vitro. The source of neurotrophins may comprise the innervated site into which the composition is placed in an in vivo embodiment of the method. More preferably however, in both in vivo and in vitro embodiments, the source of neurotrophins comprises a solution of neurotrophins which is flowed through or over the surface of the scaffold having the located Trk or fragment. The method is preferably used for the regeneration of severed nerves in vivo.

The invention also provides a stem cell, nerve progenitor cell, neuronal cell or tissue obtained or obtainable by a method according to the fourth aspect of the invention.

In a fifth, and related, aspect, the present invention provides a method of transplanting stem cells, nerve progenitor cells, nerve cells or tissue, the method comprising taking a sample of donor stem cells, nerve progenitor cells or nerve cells from a suitable donor culture or subject; growing, regenerating or repairing the donor cells in contact with a composition according to the first aspect of the invention having Trk-neurotrophin complexes on or adjacent the surface of the scaffold; and placing the donor cells and composition into a recipient subject in need of such donor cells.

The donor and recipient subjects may be the same (i.e. an autologous graft) or different (i.e. an heterologous graft) individuals.

The compositions, methods and uses of the invention described so far avoid, at least in part, several of the shortfalls associated with prior art technology in this field. Such shortfalls include, in the case of peripheral nerve repair using autograft material, donor site morbidity, inadequate return of function and aberrant regeneration. The use of synthetic biodegradable polymers in conjunction with Schwann cells is limited by the need for additional rounds of cell culture and by the necessity of the incorporation of cells into the site to be treated. Nerve transplants are to be avoided if possible since they may expose the patient to an increased risk of variant-Creutzfelt-Jakob disease. The prior art relating to biodegradable polymer surfaces presenting RGD-type peptides provides a method of directing cell spreading and regeneration but does not address how vital growth factors can be provided, especially in an in vivo setting. The growth factors required by growing, repairing and/or regenerating neurones need to be available at the neuronal cell surface for uptake. Using the present invention, it is possible to form patterns of Trk or fragments on or adjacent a scaffold surface and thereby to hold one or more of a variety of neurotrophins for presentation to growing neurones.

In a sixth aspect of the present invention there is provided a biocompatible, biodegradable composition for controlled release of a Trk or fragment or homologue thereof, the composition comprising a reservoir, formed from a biodegradable and biocompatible material, and a Trk, or a neurotrophin-binding fragment or homologue thereof, intimately associated with the reservoir and/or located at or adjacent a surface of the reservoir.

The reservoir may have any of the preferred features of the scaffold described above. The Trk may be bound to the reservoir surface as described above. The composition may contain both surface-bound Trk or fragments and Trk or fragments embedded within the material of the reservoir. Such a mixed system may provide greater flexibility in the control of Trk release rates. The composition may be suitable for in vitro and/or in vivo use.

The invention also provides a composition according to the sixth aspect, for use in therapy.

In a seventh and related aspect, the invention provides the use of a Trk, or a neurotrophin-binding fragment or homologue thereof in the preparation of a controlled release medicament for the treatment of a condition associated with elevated neurotrophin levels, the medicament comprising a reservoir formed from a biodegradable and biocompatible material, and the Trk or fragment or homologue being intimately associated with the reservoir and/or located at or adjacent a surface of the reservoir.

The invention also provides a method of treatment of a condition associated with elevated neurotrophin levels in a subject, the method comprising the administration to the subject of a composition according to the sixth aspect of the invention.

The condition to be treated may be Alzheimer's disease or may be a pain disorder. The pain may be a symptom of idiopathic sensory urgency (ISU), interstitial cystitis, arthritis, shingles, peripheral inflammation, chronic inflammation, an oncological condition or postherpetic neuralgia.

In an eighth aspect, the invention provides a biocompatible, biodegradable composition for encouraging controlled growth, regeneration or repair of biological tissue or cells, the composition comprising a scaffold, formed from biodegradable and biocompatible material, and a receptor for a growth factor, or a growth factor-binding fragment or homologue thereof, located at or adjacent a surface of the scaffold.

The growth factor may be a neurotrophin.

Furthermore, in a ninth aspect, the invention provides a biocompatible, biodegradable composition for encouraging controlled growth, regeneration or repair of biological tissue or cells, the composition comprising a scaffold, formed from biodegradable and biocompatible material, and a growth factor, or a functional fragment or homologue thereof, located at or adjacent a surface of the scaffold. The growth factor, which may be a neurotrophin, may be covalently or non-covalently bound to the scaffold. The invention also provides a composition according to the ninth aspect, for use in therapy.

The invention will be now described in more detail by way of example only and with reference to the appended drawings, of which:

FIG. 1 shows a schematic representation of the TrkA structure;

FIG. 2 provides the nucleotide and derived amino acid sequence of TrkAIg2, including the N-terminal six-His tag and the six amino acid insert VSFSPV (underlined);

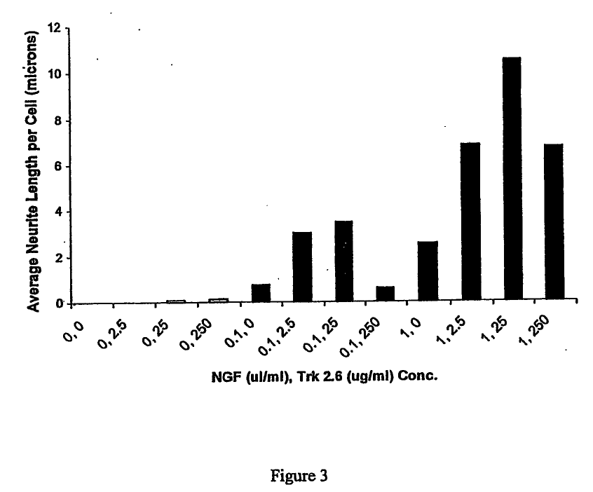

FIG. 3 illustrates the results of an experiment looking at the in vitro effects of Trk- and NGF-modified surfaces on neurite growth;

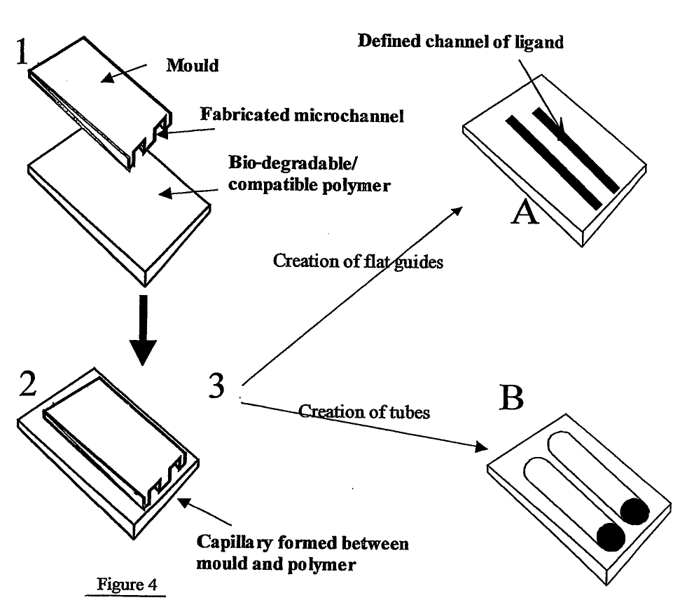

FIG. 4 illustrates, schematically, a protocol for the production of a tissue regeneration scaffold comprising either patterned channels of ligand or tubes lined with ligands;

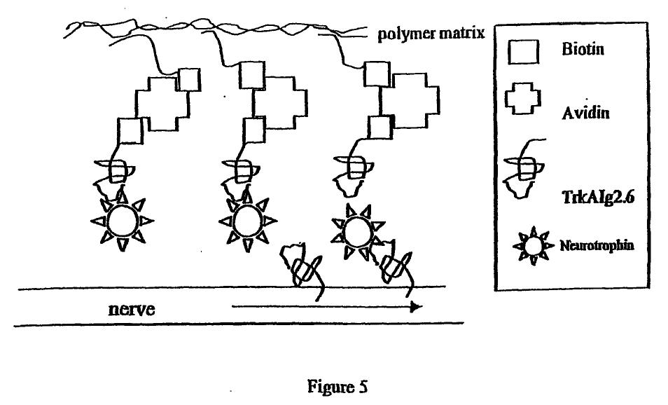

FIG. 5 shows a simplified, partial cross-sectional structural representation of a composition according to the present invention; and

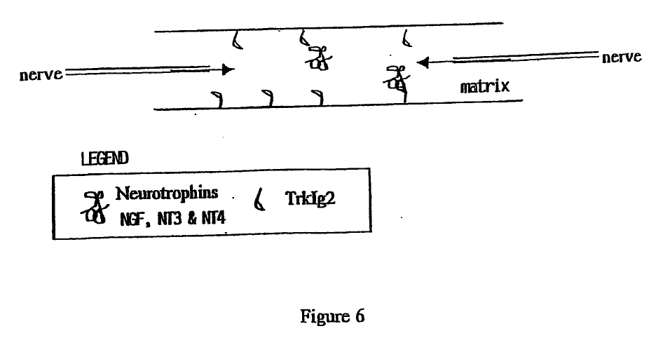

FIG. 6 illustrates how the composition of the present invention may be used to encourage neuronal growth and extension.

### EXAMPLE 1

### Structure of TrkAIg₂ and TrkAIg2.6

TrkA and isolated domains thereof are further described in WO99/53055, the disclosure of which is incorporated by reference. The accompanying FIG. 1 illustrates its structure schematically (also Robertson et al (2001) BBRC, 282: 131). The filled circles represent glycosylation sites. TrkAIg2 is defined in this example as including Ig-like subdomain 2 and the proline rich region. The sequence (TrkAIg2.6-6His) shown in FIG. 2 shows the nucleotide sequence and derived amino acid sequence of TrkAIg2 with 6×His tag. Sequence from human TrkA is in bold, 6 amino acid insert variant is underlined. This sequence includes the human TrkA sequence (amino acids 22 to 150) and a flanking sequence from the pET15b vector (amino acids 1 to 21) which also codes for an N-terminal 6×His tag. The vector sequence (codons 452 to 468, FIG. 2) also provides for a stop codon. The putative extracellular domain of human TrkA is taken to be either 375 or 381 amino acids long depending on whether the 6 amino acid insert VSFSPV is present.

It has recently been shown that a protein consisting of the two immunoglobulin-like domains and proline-rich region alone are able to bind NGF with a similar affinity to that of the complete extracellular domain (Holden et al (1997) Nature Biotechnology, 15: 668). This region is defined here as TrkAIg1,2. In addition, it has been found that an even smaller domain of TrkA, referred to as TrkAIg2 (shown in FIG. 2 as amino acids 22 to 150) able to bind NGF with a similar affinity to the complete extracellular domain or the TrkAIg1,2 region and is thus primarily responsible for the binding properties of these larger entities. TrkAIg2 which contains the six amino acid insert VSFSPV, as shown in FIG. 2 as amino acids 130 to 135, is referred to here as TrkAIg2.6.

### EXAMPLE 2

### Neuronal Growth Enhancement by Immobilisation of NGF Using Trk

This study demonstrates the feasibility of using a Trk fragment to immobilise NGF to a biomaterial surface and thus provide a localised environment to stimulate peripheral nerve regeneration. Experiments were performed using PLA-PEG-biotin as a base material, which has previously been demonstrated to enable facile surface patterning of ligands to spatially control tissue regeneration (WO99/36107). In this Example, the Trk fragment used was the 6-His tagged version of TrkIgA2.6 (TrkIgA2.6-6His).

PC12 cells were grown in RPMI-1640 media, supplemented with 10% horse serum, 5% foetal calf serum, antibiotic/antimycotic, and L-glutamine, at a density of 2-5×10⁵ cells/ml, Each T75 flask containing between 10-20 ml of media.

As PC12 cells require surface attachment in order to enable neurite extension, their non-adherence to tissue culture plastic means that surfaces must be precoated with extracellular matrix substrates such as collagen or laminin. T-75 flasks and 24 well plates were collagen coated by leaving a 0.01% collagen Type IV solution in distilled sterile water on the surface for two hours (10 ml and 0.5 ml respectively) and then air drying overnight.

Prior to the TrkAIg2.6 neurite extension studies, PC-12 cells grown upon collagen-coated flasks were primed with NGF (50 ng/ml) for 5 days, fresh media and NGF being added every 24 hours.

TrkA2.6-6His (in 20 mM Sodium Phosphate buffer pH 8.0, 100 mM Sodium Chloride and 10% glycerol) was prepared using the method of WO99/53055 and pending application number PCT/GB02/04214 and a Sigma kit was used to biotinylate using standard procedures. A stock solution of approximately 250 μg/ml was prepared.

PLA-PEG-biotin coated Iwaki Non-Treated 24 well plates were prepared using 0.25 ml of 2 mg/ml polymer dissolved in 2,2,2-Trifluoroethanol (TFE), dropcast onto well plates & dried in oven at 60° C. for 1 hour. Plates were then washed in PBS & stored in a refrigerator overnight. Three test plates were prepared for each batch of biotinylated TrkAIg2.6.

Avidin was attached to the PLA-PEG-biotin-coated plates using 0.5 ml of a 500 μg/ml solution in distilled water for 45 mins at 37° C., before again washing the plates with PBS.

1 ml of the biotinylated TrkA prepared above was then added to the plates at a conc. of between 0-250 μg/ml in distilled water for 1 hour at 37° C. Plates were then washed with PBS. 0.5 ml of 0-50 ng/l (=0-1 μl/ml) of NGF in PBS was then added for 45 mins at 37° C. before a final PBS wash. This was then followed by coating of all wells with 0.25 ml of 0.0025% Collagen Type IV in distilled water for 1 hour before washing. Control wells contained NGF media in the presence of PLA-PEG-Biotin or 0.01% collagen, but no TrkAIg2.6.

Cells (passage 18) were seeded at 2.0×10⁴ cells/ml per well. The media was replaced with a fresh supply (containing 0, 0.1 or 1 μl NGF) after 24 hours.

After allowing the cells to attach and extend neurites over a 48-hour period in an incubator set at 37° C./5% CO₂, the media was aspirated to remove any loosely adherent cells and again replaced. Images were then taken using a Nikon Eclipse TS100 microscope and DN100 digital camera at × magnification with a 0.45× C-mount. Process length and cell number were measured using Leica Qwin image analysis software.

FIG. 3 shows neurite extension from the PC12 nerve cell line following culture upon a range of Trk-, and subsequently, NGF-modified surfaces. The data shows an increase in neurite extension with increasing amounts of surface-immobilised NGF, and that an optimal Trk concentration appears to be within the range used.

This study illustrates the ability to induce neurite outgrowth from cells cultured upon modified PLA-PEG-biotin surfaces, by presenting NGF using a receptor fragment to attach the growth factor to the surface. Extracellular matrix molecules are also preferably presented at the surface in order to enhance cell surface attachment.

Within this data, a Trk concentration-dependant effect can be seen at each different NGF concentration and it appears that an optimum Trk concentration exists. For TrkAIg2.6, this may be between 2.5 and 250 μg/ml, depending on experimental conditions, and may be around 25 μg/ml. The decrease in neurite outgrowth at the higher concentration may be due to toxicity effects or increased competition for NGF between immobilised receptor fragments and the cells themselves.

### EXAMPLE 3

### A Nerve Regeneration Scaffold

FIG. 4 shows schematically how a scaffold suitable for a composition according to the invention may be generated. The scaffold may be fabricated to comprise patterned channels of ligand (A) or to comprise tubes which have a patterned lining of ligand (B). FIG. 5 shows schematically how a composition according to the invention may be assembled. Briefly, the polymer scaffold or matrix of FIG. 4 (polyester, such as poly(lactic acid), poly(lactic-co-glycolic) acid or block copolymers of these polyesters with PEG) is patterned (as described in WO99/36107) with biotin molecules. Avidin is then introduced to the scaffold to produce an avidin-patterned surface. Biotin-labelled TrkAIg2.6 is then passed through the scaffold; the biotin labels bind to unoccupied binding sites on the avidin molecules and thus produce a Trk-patterned surface. Finally, neurotrophins are introduced; these bind to the TrkAIg2.6 and thus produce a neurotrophin-patterned, biodegradable polymer scaffold.

In the example shown in FIG. 5 (and more clearly in FIG. 6), the scaffold has a structure comprising a number of tubes or conduits, of which one is shown. Nerve cells are able to grow along the lumen of the tube/conduit, obtaining neurotrophins from the prepared surface as they do so. In FIG. 5, it can be seen that the neurotrophins are taken up by the nerve cells by means of the Trk molecules expressed on the surface of the cells. The composition of the invention may be used, in particular, in nerve regeneration following acute spinal injury, acute peripheral injury and chronic injury.

### EXAMPLE 4

### A controlled Release Formulation of Trk

An implantable polymeric (poly(lactic-co-glycolic)acid) reservoir of TrkAIg2.6 was prepared as described in Example 3 in relation to the neuronal repair scaffold but with the exclusion of the final step of adding neurotrophins. The reservoir was implanted in an in vivo model of neuropathic pain. Prolonged release of Trk and resulting analgesia were observed.

1. A biocompatible, biodegradable composition for encouraging controlled neuronal growth, regeneration or repair, the composition comprising a scaffold, formed from biodegradable and biocompatible material, and a tyrosine receptor kinase (Trk), or a neurotrophin-binding fragment or homologue thereof, located at or adjacent a surface of the scaffold.

2. A composition according to claim 1 wherein the Trk is TrkA, B or C, an alternatively spliced version thereof, a pan-Trk, a functional homologue of a Trk or a combination of Trk types.

3. A composition according to claim 1 wherein the fragment of the Trk comprises an Ig-like sub-domain.

4. A composition according to claim 3 wherein the fragment comprises Ig-like sub-domain 2 of TrkA.

5. A composition according to claim 4 wherein the Ig-like sub-domain 2 includes the amino acid insert VSFSPV.

6. A composition according to claim 5 wherein the fragment comprises the sequence shown in FIG. 2 or a functional homologue thereof.

7. A biocompatible, biodegradable composition for encouraging controlled neuronal growth, regeneration or repair, the composition comprising a scaffold, formed from biodegradable and biocompatible material and a tyrosine receptor kinase (Trk), or a neurotrophin-binding fragment or homologue thereof located at or adjacent a surface of the scaffold and including one or more types of neurotrophin bound to the Trk or fragment or homologue.

8. A composition according to claim 7 wherein the neurotrophin is selected from NGF, BDNF, NT3 and NT4.

9. A composition according to claim 1 or claim 7 including one or more extracellular matrix components located at or adjacent a surface of the scaffold.

10. A composition according to claim 9 wherein the extracellular matrix components include collagen.

11. A composition according to claim 9 wherein the extracellular matrix components comprise peptides containing the sequences RGD, YIGSR and/or IKVAV.

12. A composition according to claim 1 or claim 7 wherein the material of the scaffold is a biodegradable and biocompatible polymer.

13. A composition according to claim 12 wherein the polymer is selected from polyhydroxy acids, polysaccharides, poly (amino acids), poly (pseudo amino acids), and copolymers prepared from the monomers of any of these polymers.

14. A composition according to claim 13 wherein the polymer is a block copolymer with a poly (alkylen glycol).

15. A composition according to claim 14 wherein the polymer is a block copolymer of poly (ethylene glycol) with poly (lactic acid), poly (glycolic acid) or poly (lactic-co-glycolic) acid.

16. A composition according to claim 1 or claim 7 wherein the Trk or fragment or homologue is located at or adjacent the surface of the scaffold by means of one or more specific molecular interactions and the material of the scaffold is a biodegradable and biocompatible polymer.

17. A composition according to claim 16 wherein the one or more specific molecular interactions take place between one or more anchor molecules bound to or adjacent the scaffold surface and one or more tag molecules bound to the Trk or fragment or homologue.

18. A composition according to claim 17 wherein a tag molecule is biotin and an anchor molecule is avidin or streptavidin, or vice versa.

19. A composition according to claim 17 wherein an adapter molecule is used which is capable of simultaneously binding to both the tag and the anchor.

20. A composition according to claim 19 wherein both the tag and the anchor are biotin and the adapter is avidin or streptavidin.

21. A composition according to claim 1 or claim 7 wherein the scaffold is tubular in shape.

22. A composition according to claim 1 or claim 7 wherein the Trk is present at a concentration of around 2.5 to 250 μg/ml.

23. A biocompatible, biodegradable composition for encouraging controlled growth, regeneration or repair of biological tissue or cells, the composition comprising a scaffold, formed from biodegradable and biocompatible material, and a receptor for a growth factor, or a growth factor-binding fragment or homologue thereof, located at or adjacent a surface of the scaffold.

24. A composition according to claim 23 wherein the growth factor is a neurotrophin.

25. A composition according to claim 1, 7, or 23 wherein the Trk or other growth factor receptor is patterned at or adjacent the surface of the scaffold so as to provide directional control of growth, regeneration or repair of the neuronal or other biological tissue or cells.

26-27. (canceled)

28. A method of encouraging nerve growth, regeneration or repair, the method comprising contacting a composition according to any of claims 1 or 7 with a source of neurotrophins so as to form Trk-neurotrophin complexes on or adjacent the surface of the scaffold, contacting the composition with a stem cell, nerve progenitor cell, neuronal cell or tissue and allowing the stem cell, nerve progenitor cell, neuronal cell or tissue to grow, regenerate or repair upon or adjacent the surface of the scaffold.

29. A method of transplanting stern cells, nerve progenitor cells, nerve cells or tissue, the method comprising taking a sample of donor stem cells, nerve progenitor cells, or nerve cells from a suitable donor culture or subject; growing, regenerating or repairing the donor cells in contact with a composition according to any of claims 1 or 7 having Trk-neurotrophin complexes on or adjacent the surface of the scaffold; and placing the donor cells and composition into a recipient subject in need of such donor cells.

30. A biocompatible, biodegradable composition for controlled release of a Trk or fragment or homologue thereof, the composition comprising a reservoir, formed from a biodegradable and biocompatible material, and a Trk, or a neurotrophin-binding fragment or homologue thereof, intimately associated with the reservoir and/or located at or adjacent a surface of the reservoir.

31. (canceled)

32. A method of treating a condition associated with elevated neurotrophin levels, comprising administering a composition according to claim 30 to a subject in need thereof.

33. The method of claim 32 wherein the condition to be treated is Alzheimer's disease or a pain disorder.

34. The method of claim 33 wherein the pain disorder is associated with idiopathic sensory urgency, interstitial cystitis, arthritis, shingles, peripheral inflammation, chronic inflammation, an oncological condition or postherpetic neuralgia.

35. A stem cell, nerve progenitor cell, neuronal cell or tissue obtained a method according to claim 28.

36. A biocompatible, biodegradable composition for encouraging controlled growth, regeneration or repair of biological tissue or cells, the composition comprising a scaffold, formed from biodegradable and biocompatible material, and a growth factor, or a functional fragment or homologue thereof, located at or adjacent a surface of the scaffold.

37. (canceled)

38. A composition according to claim 2 wherein the fragment of the Trk comprises an Ig-like sub-domain.

39. A composition according to claim 38 wherein the fragment comprises Ig-like sub-domain 2 of TrkA.

40. A composition according to claim 39 wherein the Ig-like sub-domain 2 includes the amino acid insert VSFSPV.

41. A composition according to claim 40 wherein the fragment comprises the sequence shown in FIG. 2 or a functional homologue thereof.

42. A composition according to claim 38 including one or more types of neurotrophin bound to the Trk or fragment or homologue.

43. A composition according to claim 10 wherein the extracellular matrix components comprise peptides containing the sequences RGD, YIGSR and/or IKVAV.

44. A composition according to claim 43 wherein the material of the scaffold is a biodegradable and biocompatible polymer.

45. A composition according to claim 44 wherein the polymer is selected from polyhydroxy acids, polysaccharides, poly (amino acids), poly (pseudo amino acids), and copolymers prepared from the monomers of any of these polymers.

46. A composition according to claim 45 wherein the polymer is a block copolymer with a poly (alkylen glycol).

47. A composition according to claim 46 wherein the polymer is a block copolymer of poly (ethylene glycol) with poly (lactic acid), poly (glycolic acid) or poly (lactic-co-glycolic) acid.

48. A composition according to claim 43 wherein the Trk or fragment or homologue is located at or adjacent the surface of the scaffold by means of one or more specific molecular interactions.

49. A composition according to claim 48 wherein the one or more specific molecular interactions take place between one or more anchor molecules bound to or adjacent the scaffold surface and one or more tag molecules bound to the Trk or fragment or homologue.

50. A composition according to claim 49 wherein a tag molecule is biotin and an anchor molecule is avidin or streptavidin, or vice versa.

51. A composition according to claim 49 wherein an adapter molecule is used which is capable of simultaneously binding to both the tag and the anchor.

52. A composition according to claim 51 wherein both the tag and the anchor are biotin and the adapter is avidin or streptavidin.

53. A composition according to claim 43 wherein the scaffold is tubular in shape.

54. A composition according to claim 43 wherein the Trk is present at a concentration of around 2.5 to 250 μg/ml.
